(19) United States
(12) Patent Application Publication
(10) Pub. No.: US 2025/0214994 A1
(43) Pub. Date: Jul. 3, 2025

(54) PROCESS FOR THE PREPARATION OF PALBOCICLIB

(71) Applicant: Glenmark Life Sciences Limited, Solapur (IN)

(72) Inventors: Venkata Raghavendra Acharyulu PALLE, Pune (IN); Pratik R. PATEL, Vadodara (IN); Srinivas Harinath HARATHI DWARAKANATH, Vikarabad (IN); Shivaji Haribhau SHELKE, Thane (IN); Sachin Mahadeo LAD, Thane (IN); Girish Tilakchand PATLE, Pune (IN)

(21) Appl. No.: 18/853,801

(22) PCT Filed: Apr. 3, 2023
(86) PCT No.: PCT/IB2023/053343

§ 371 Date: Oct. 3, 2024

(30) Foreign Application Priority Data
Apr. 6, 2022 (IN) 202221020699

(51) Int. Cl. C07D 471/04

(52) U.S. Cl. CPC C07D 471/04

(57) ABSTRACT

The present invention relates to an improved process for the preparation of palbociclib. The present invention also relates to a novel crystalline Form G of tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (also known as N-BOC palbociclib), and a process for its preparation. The novel Form G of tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (also known as N-BOC palbociclib) is substantially in accordance with FIG. 1.

### PRIORITY

This application claims the benefit of Indian Provisional Applications 202221020699 filed on Apr. 6, 2022, entitled “Crystalline N-BOC Palbociclib and its use in the process for the preparation of Palbociclib” the contents of which are incorporated herein by reference.

### FIELD OF THE INVENTION

The present invention relates to an improved process for the preparation of palbociclib. The present invention also relates to a novel crystalline Form G of tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (also known as N-BOC palbociclib), and a process for its preparation. Further, the present invention relates to a pharmaceutical composition containing a therapeutically effective amount of palbociclib, which is prepared as described herein.

### BACKGROUND OF THE INVENTION

Palbociclib, known by its chemical name, 6-acetyl-8-cyclopentyl-5-methyl-2-{[5-(piperazin-1-yl)pyridin-2-yl]amino}pyrido[2,3-d]pyrimidin-7(8H)-one, is represented by the structure of formula I (the “compound I”).

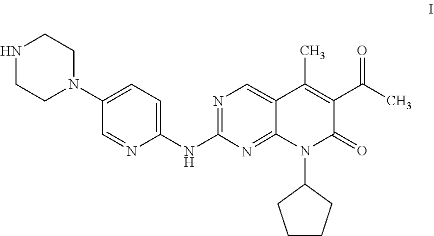 [SMILES: CC(=O)c1c(C)c2cnc(Nc3ccc(N4CCNCC4)cn3)nc2n(C2CCCC2)c1=O]

Palbociclib is described in published PCT application no. WO2003062236 (herein after referred to as WO'236).

Palbociclib, sold under the trade name of IBRANCE® is indicated for the treatment of adult patients with hormone receptor (HR)-positive, human epidermal growth factor receptor 2 (HER2)-negative advanced or metastatic breast cancer in combination with: 1) an aromatase inhibitor as initial endocrine-based therapy in postmenopausal women or in men; or 2) fulvestrant in patients with disease progression following endocrine therapy.

Various processes for the synthesis of Palbociclib are known in the art, for instance the U.S. Pat. No. 9,850,244 (US'244 patent) and the Journal article of Organic Process Research Development (2016), volume 20, page no. 1191-1202 describe processes for the preparation of this compound.

### SUMMARY OF THE INVENTION

The present invention relates to a process for the preparation of palbociclib, a compound of formula I (the “compound I”),

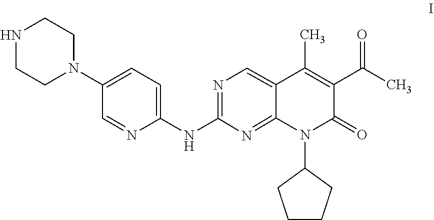 [SMILES: CC(=O)c1c(C)c2cnc(Nc3ccc(N4CCNCC4)cn3)nc2n(C2CCCC2)c1=O]

comprising the steps of.
a) reacting 6-bromo-2-chloro-8-cyclopentyl-5-methylpyrido[2,3-d]pyrimidin-7(8H)-one, a compound of formula II (the “compound II”) with tert-butyl 4-(6-amino-3-pyridyl) piperazine-1-carboxylate, a compound of formula III (the “compound III”) in the presence of a Grignard reagent,

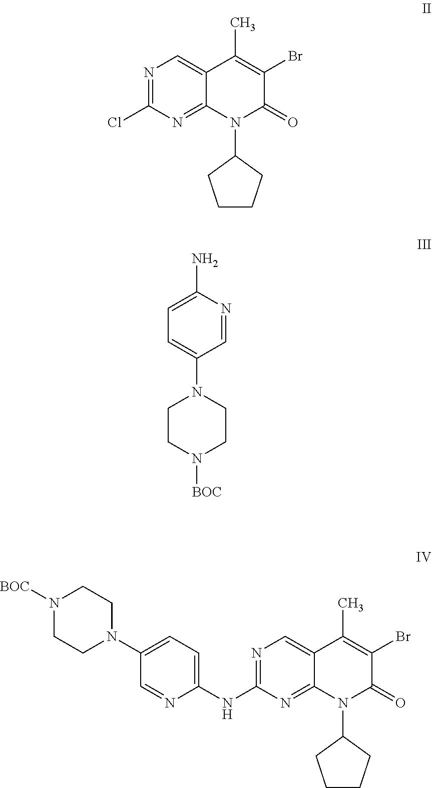 [SMILES: Cc1c(Br)c(=O)n(C2CCCC2)c2nc(Cl)ncc12 ; CC(C)(C)OC(=O)N1CCN(c2ccc(N)nc2)CC1 ; Cc1c(Br)c(=O)n(C2CCCC2)c2nc(Nc3ccc(N4CCN(C(=O)OC(C)(C)C)CC4)cn3)ncc12]

wherein BOC refers to tert-butoxycarbonyl;
to obtain 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid tert-butyl ester, a compound of formula IV (the “compound IV”), wherein the Grignard reagent is added in one lot, and the compound II is added in at least two portions;
b) reacting the compound IV obtained in the step a) with n-butyl vinyl ether, a compound of formula V (the “compound V”);

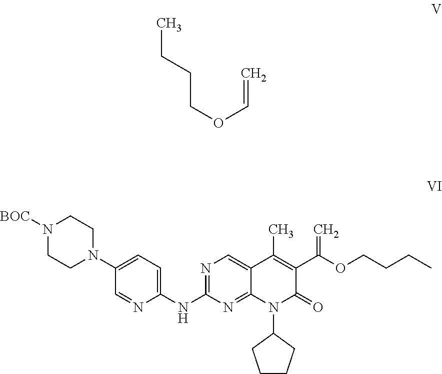

to obtain tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate, a compound of formula VI (the “compound VI” or N-BOC palbociclib); and c) reacting the compound VI obtained in the step b) with an acid followed by treatment with a base to obtain palbociclib, the compound 1.

The present invention further relates to crystalline tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)-piperazine-1-carboxylate (N-BOC palbociclib) designated as Form G, which is characterised by X-ray powder diffraction pattern (XRPD) having peaks at about 5.87, 9.29, 10.52, 11.77, 12.97, 13.72, 16.31, and 22.44±0.2 degrees 2 theta.

The present invention also relates to a process for the preparation of crystalline Form G of N-BOC palbociclib (the compound VI), comprising the steps of:
(i) providing a solution of N-BOC palbociclib, compound VI, in an ester solvent;
(ii) heating the solution of the step (i);
(iii) optionally, filtering the hot solution of the step (ii);
(iv) cooling the solution of the step (ii) or the step (iii); and
(v) isolating the crystalline Form G of N-BOC palbociclib obtained in the step (iv), wherein the Form G is characterised by XRPD having peaks at about 5.87, 9.29, 10.52, 11.77, 12.97, 13.72, 16.31, and 22.44±0.2 degrees 2 theta.

The present invention further provides a compound of formula VII (the “compound VII”),

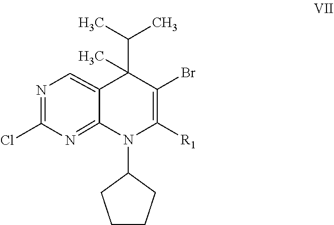

wherein R¹ is hydroxy or isopropyl.

The present invention also provides a compound of formula VIII (the “compound VIII”),

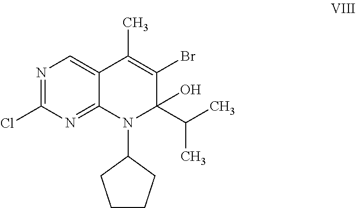 [SMILES: CC1=C(Br)C(O)(C(C)C)N(C2CCCC2)c2nc(Cl)ncc21]

The present invention also provides palbociclib, the compound I, having purity of at least 99% w/w as determined by HPLC (High Performance Liquid Chromatography).

Further, the present invention provides palbociclib having a content of compound VII, compound VIII and compound IX

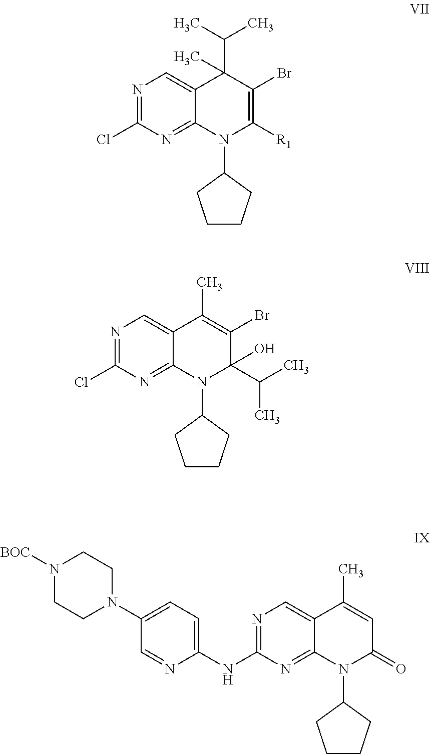

in an amount ranging from about 0.15% w/w to about 0.03% w/w as determined by HPLC (High Performance Liquid Chromatography).

The present invention also provides a pharmaceutical composition comprising palbociclib (compound I), wherein the compound I is prepared using crystalline Form G of N-BOC palbociclib, wherein the content of the compound VII, compound VIII and the compound IX is less than 1% w/w as measured by HPLC.

### BRIEF DESCRIPTION OF THE DRAWINGS

FIG. 1 is a characteristic X-ray Powder Diffraction Pattern (XRPD) of crystalline Form G of N-BOC palbociclib as obtained in Example 2.

### DETAILED DESCRIPTION OF THE INVENTION

The present invention relates to a process for the preparation of palbociclib, a compound of formula I (the “compound I”),

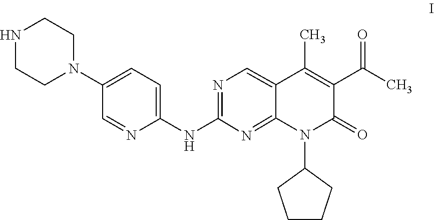 [SMILES: CC(=O)c1c(C)c2cnc(Nc3ccc(N4CCNCC4)cn3)nc2n(C2CCCC2)c1=O]

comprising the steps of:
a) reacting 6-bromo-2-chloro-8-cyclopentyl-5-methylpyrido[2, 3-d]pyrimidin-7(8H)-one, a compound of formula II, (the “compound II”) with tert-butyl 4-(6-amino-3-pyridyl) piperazine-1-carboxylate, a compound of formula III (the “compound III”) in the presence of a Grignard reagent,

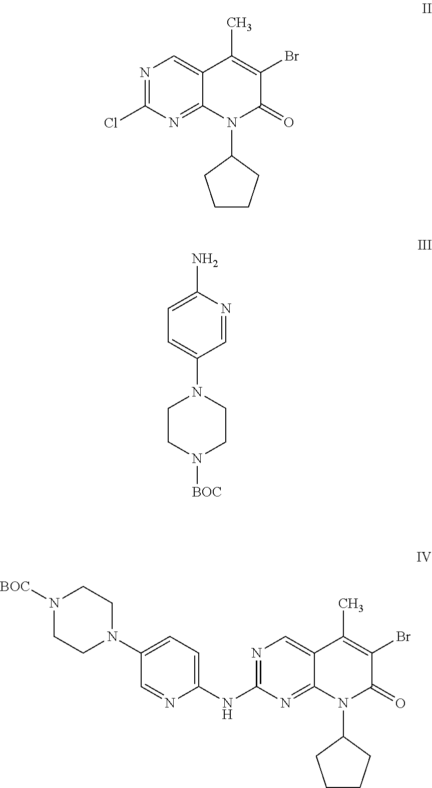 [SMILES: Cc1c(Br)c(=O)n(C2CCCC2)c2nc(Cl)ncc12 ; CC(C)(C)OC(=O)N1CCN(c2ccc(N)nc2)CC1 ; Cc1c(Br)c(=O)n(C2CCCC2)c2nc(Nc3ccc(N4CCN(C(=O)OC(C)(C)C)CC4)cn3)ncc12]

to obtain 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid butyl ester, a compound of formula IV (the “compound IV), wherein the Grignard reagent is added in one lot, and the compound II is added in at least two portions;
b) reacting the compound IV obtained in the step a) with n-butyl vinyl ether, a compound of formula V (the “compound V”);

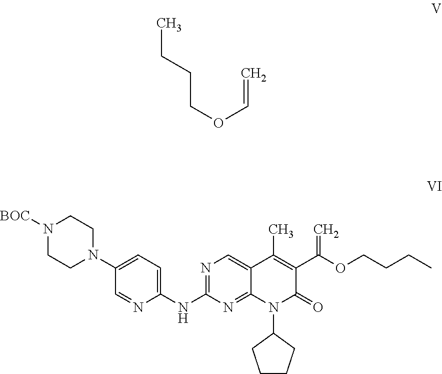

to obtain tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl) piperazine-1-carboxylate, a compound of formula VI (the “compound VI”); and
c) reacting the compound VI obtained in the step b) with an acid followed by treatment with a base to obtain palbociclib, the compound I.

In one embodiment, the step a) of the above process comprises the steps of.
(a-i) reacting compound II with compound III in a mixture comprising a solvent and a Grignard reagent, wherein less than or equal to one molar equivalent of compound II is used with respect to compound III, and wherein the Grignard reagent is added in one lot;
(a-ii) stirring the reaction mixture of step (a-i) for a period of about 15 minutes to about 1 hour;
(a-iii) adding second portion of the compound II to the mixture of step (a-ii) to obtain compound IV;
(a-iv) optionally, repeating step (a-ii) and step (a-iii) to have total molar equivalent of compound II with respect to compound III stoichiometrically between 0.8 to 1.0 molar equivalents; and
(a-v) isolating the compound IV obtained in the step (a-iii) or the step (a-iv).

In one embodiment, the step (a-i) comprises addition of Grignard reagent at a temperature below 40° C.

Inventors of the present invention carried out the process for the preparation of the compound IV under varying reaction conditions, and found that when the Grignard reagent is added in one single lot in step (a-i), the formation of undesired impurities are reduced.

In an embodiment, the reaction involving coupling of the compound II with the compound III is carried out in such a way that the compound II, prior to its addition to the reaction mixture of compound III and Grignard reagent, is not contacted with any solvent. Such an addition of compound II in the reaction mixture improves volume efficiency of the coupling reaction. The temperature at which the process of the step (a) is carried out is maintained below 40° C. as it was observed that the impurity generation is high when the temperature of the reaction mixture comprising compound II, compound III and Grignard reagent in a solvent is raised to a temperature above 40° C. It was observed that with an increase in the reaction temperature, the formation of impurities also increases. Therefore, the reaction in step (a) involving the preparation of compound IV is carried out at a temperature below 40° C. Preferably the reaction is carried out between a temperature range from about 0° C. to about 35° C. More preferably, the reaction is carried out between a temperature range from about 15° C. to about 25° C.

In the context of the present invention, the step (a) may be carried out at a temperature ranging from about 0° C. to about 35° C. or about 15° C. to about 25° C.

In one embodiment, in the above step a), the compound II may be added to the reaction mixture comprising compound III and Grignard reagent, in the presence of a solvent.

In another embodiment, in the above step a), the compound II may be added to the reaction mixture comprising compound III, solvent and Grignard reagent in two portions.

In an embodiment, in the above step a), the compound II may be added to the reaction mixture comprising compound III, solvent and Grignard reagent in three portions.

In an embodiment, in the above step a), the compound II may be added to the reaction mixture comprising compound III, solvent and Grignard reagent in four portions.

The solvent used in the step a) includes, but is not limited to, hydrocarbons such as toluene, xylene, chlorobenzene, heptane, hexane, cyclohexane and the like; alcohols such as methanol, ethanol, n-propanol, t-butanol, n-5 butanol, isopropanol and like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, butyronitrile, propionitrile, benzonitrile and the like; amides such as dimethylformamide, dimethyl acetamide and the like; sulfoxides such as dimethyl sulfoxide; water or mixtures thereof.

The Grignard reagent used in step a) is selected from the group consisting of isopropyl magnesium chloride, cyclopropyl magnesium chloride, cyclohexyl magnesium chloride, cyclopentyl magnesium chloride, t-butyl magnesium chloride, p-tolyl magnesium chloride, hexyl magnesium chloride, isopentyl magnesium chloride, and cyclohexyl magnesium bromide.

In one embodiment, the present invention provides a compound of formula VII (the “compound VII”),

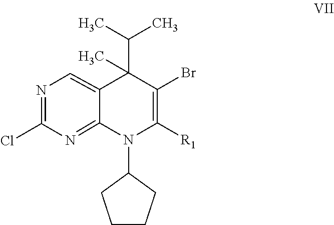

wherein R¹ is hydroxy or isopropyl.

In one embodiment, the present invention provides a compound of formula X (the “compound X”), when R is isopropyl in compound VII

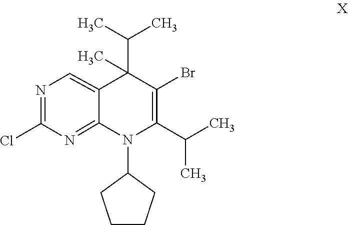 [SMILES: CC(C)C1=C(Br)C(C)(C(C)C)c2cnc(Cl)nc2N1C1CCCC1]

In one embodiment, the present invention provides a compound of formula XI (the “compound XI”), when R is hydroxyl in compound VII,

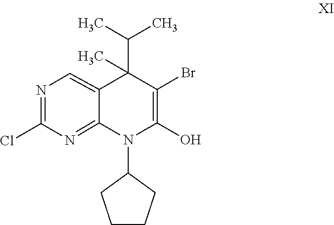 [SMILES: CC(C)C1(C)C(Br)=C(O)N(C2CCCC2)c2nc(Cl)ncc21]

In one embodiment, the present invention provides a compound of formula VIII (the “compound VIII”)

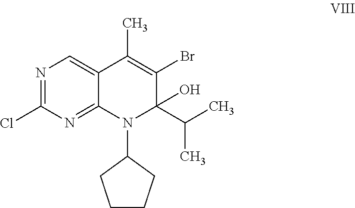 [SMILES: CC1=C(Br)C(O)(C(C)C)N(C2CCCC2)c2nc(Cl)ncc21]

Inventors of the present invention have identified the compounds VII and VIII as the impurities present in palbociclib, the compound I.

It was observed that in step a) of the above process, when the compound II was added in one lot or the Grignard reagent was added in portions, the contents of the undesired compound VII, the compound VIII, and a des bromo compound of formula IX (the “compound IX”) are high, thereby resulting in reduction of the yield and purity of N-BOC palbociclib, and ultimately the reduction of the yield and purity of the desired final compound, palbociclib, the compound I. The compound IX is structurally represented below:

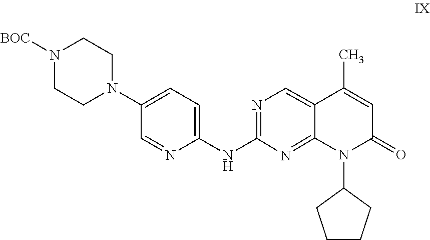 [SMILES: Cc1cc(=O)n(C2CCCC2)c2nc(Nc3ccc(N4CCN(C(=O)OC(C)(C)C)CC4)cn3)ncc12]

In one embodiment, in the step a) of the above process, the compound II is reacted with the compound III in at least two portions, particularly three portions or four portions, and at less than or equal to one molar equivalent with respect to compound III. The time interval between each portion varies between from about 15 min to about 1 hour. The compound II is added in portions to the reaction mixture comprising compound III to have total molar equivalent of compound II with respect to compound III is stoichiometrically between 0.8 to 1.0 molar equivalents.

In one embodiment, the step a) of the above process involves reacting the compound II with compound III in a reaction mixture comprising a solvent and a Grignard reagent, wherein compound II is added in at least two portions and Grignard reagent is added in one lot to obtain compound IV having the contents of compound VII and compound VIII less than 1% w/w as measured by HPLC.

In one embodiment, the step a) of the above process involves reacting the compound II with compound III in a reaction mixture comprising a solvent and a Grignard reagent, wherein the compound II is added in at least two portions, the Grignard reagent is added in one lot and the reaction is carried out at a temperature below 40° C. to obtain compound IV having the contents of compound VII, the compound VIII, and the compound of formula IX less than 1% w/w as measured by HPLC.

In one embodiment, the step b) of the above process involves reacting the compound IV with the compound V in the presence of a base, a solvent and a catalyst to obtain the compound VI (N-BOC palbociclib).

In an embodiment, the solvent used in the step b) includes, but is not limited to hydrocarbons such as toluene, xylene, chlorobenzene, heptane, hexane, cyclohexane and the like; alcoholic solvents such as methanol, ethanol, n-propanol, t-butanol, n-butanol, isopropanol and like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, butyronitrile, propionitrile benzonitrile and the like; amides such as dimethylformamide, dimethyl acetamide and the like; sulfoxides such as dimethyl sulfoxide; water or mixtures thereof.

In one embodiment, the base used in the step b) may be an organic base selected from the group consisting of methylamine, dimethylamine, dipropylamine, tripropylamine, tributylamine, N, N-dimethylcyclohexylamine trimethylamine, ethylamine, propylamine, butylamine, dibutylamine, triethylamine, diisopropylethylamine, cyclopropyl amine, aniline, N,N-dimethyl aniline, N-methylpiperidine, N-methyl morpholine, N-tert-butyl benzylamine, N-benzylmethylamine and piperidine.

In one embodiment, the catalyst used in step b) may be selected from the group consisting of bis[(2-diphenylphosphino)phenyl]ether, [1,1′-bis(diphenylphosphino)ferrocene]palladium(II) dichloride, palladium on carbon (Pd/C), palladium acetate, palladium chloride, tetrakis (triphenylphosphine)palladium(Pd(PPh₃)₄), tris (dibenzylideneacetone)-dipalladium[Pd₂(dba)₃], bis(dibenzylideneacetone)-palladium(0) (Pd(dba)₂), bis (benzonitrile) dichloropalladium (II) ((C₆H₅CN)₂PdCl₂).

In one embodiment, the step c) of the above process involves reacting compound VI with an acid followed by treatment with a base in the presence of a solvent to obtain palbociclib, the compound I.

Palbociclib obtained in the step c) is isolated by methods such as filtration, centrifugation and the like.

The solvent used in the step c) includes, but is not limited to hydrocarbons such as toluene, xylene, chlorobenzene, heptane, hexane, cyclohexane and the like; alcohols such as methanol, ethanol, n-propanol, t-butanol, n-5 butanol, isopropanol and like; ketone such as acetone, methyl isobutyl ketone, methyl ethyl ketone, acetone, cyclohexanone and the like; ethers such as dimethyl ether, diethyl ether, diisopropyl ether, tert-butyl methyl ether, dibutyl ether, dimethoxyethane, diethoxyethane, tetrahydrofuran, dioxane and the like; halogenated hydrocarbons such as dichloromethane, dichloroethane, chloroform, ethylene dichloride, and the like; esters such as methyl acetate, ethyl acetate, n-propyl acetate, tert-butyl acetate and the like; nitriles such as acetonitrile, benzonitrile and the like; amides such as dimethylformamide, dimethyl acetamide and the like; sulfoxides such as dimethyl sulfoxide; water or mixtures thereof.

In one embodiment, the acid used in step c) may be selected from the group consisting of hydrochloric acid, sulfuric acid, phosphoric acid Isethionic acid, methane sulphonic acid, hydrochloric acid, sulphuric acid, trifluoroacetic acid and the like.

In one embodiment, the base may be an inorganic base selected from the group consisting of hydroxides such as sodium hydroxide, potassium hydroxide and the like; carbonates such as sodium carbonate, potassium carbonate and the like; and bicarbonates such as sodium bicarbonate, potassium bicarbonate and the like.

In one embodiment, the compound VI, the N-BOC palbociclib obtained in the above step b) is crystalline.

Accordingly, the present invention provides a crystalline tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (N-BOC palbociclib), the compound VI, designated as Form G, which is characterised by XRPD having peaks at about 5.87, 9.29, 10.52, 11.77, 12.97, 13.72, 16.31, and 22.44±0.2 degrees 2 theta.

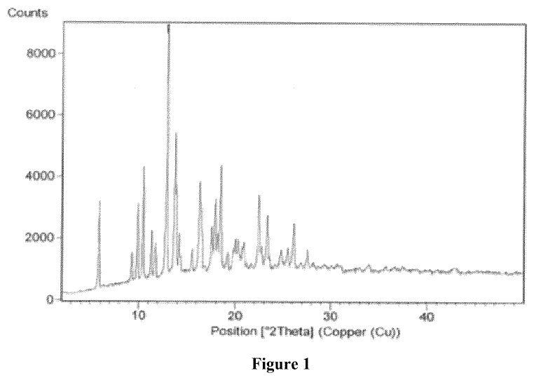

In one embodiment, the present invention provides a crystalline Form G of of tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate (N-BOC palbociclib or compound VI) as substantially illustrated in FIG. 1.

In the context of the present invention, the term “substantially illustrated” as used in reference to FIG. 1 may be understood to relate to any crystalline form of N-BOC palbociclib characterized with the graphical data having small variations, as are well known to a person skilled in the art, in comparison with the FIG. 1.

In one embodiment, the present invention provides a process for the preparation of crystalline Form G of N-BOC palbociclib, the compound VI, comprising the steps of:
(i) providing a solution of N-BOC palbociclib, the compound VI, in an ester solvent;
(ii) heating the solution of the step (i);
(iii) optionally, filtering the hot solution of the step (ii);
(iv) cooling the solution of the step (ii) or the step (iii); and
(v) isolating the crystalline Form G of N-BOC palbociclib, characterised by XRPD pattern having peaks at about 5.87, 9.29, 10.52, 11.77, 12.97, 13.72, 16.31, and 22.44±0.2 degrees 2 theta.

The ester solvent used in the step (i) may be selected from the group consisting of ethyl acetate, propyl acetate, and isopropyl acetate.

In an embodiment, the present invention provides crystalline N-BOC palbociclib, the compound VI, designated as Form G, and characterised by XRPD having peaks at about 5.87, 9.29, 10.52, 11.77, 12.97, 13.72, 16.31, and 22.44±0.2 degrees 2 theta obtained by recrystallizing from ethyl acetate.

In an embodiment, the present invention further provides palbociclib, the compound I, having a content of compound VII and a compound VIII in an amount ranging from about 0.15% w/w to about 0.03% w/w as determined by HPLC.

In one embodiment, the present invention provides Palbociclib, the compound I, having a content of compound VII, compound VIII and a compound IX in an amount ranging from about 0.15% w/w to about 0.03% w/w as determined by HPLC.

In one embodiment, the present invention provides Palbociclib, the compound I, substantially free of any of the compounds represented by the following compounds of formulae II, VI, and VIII to XV:

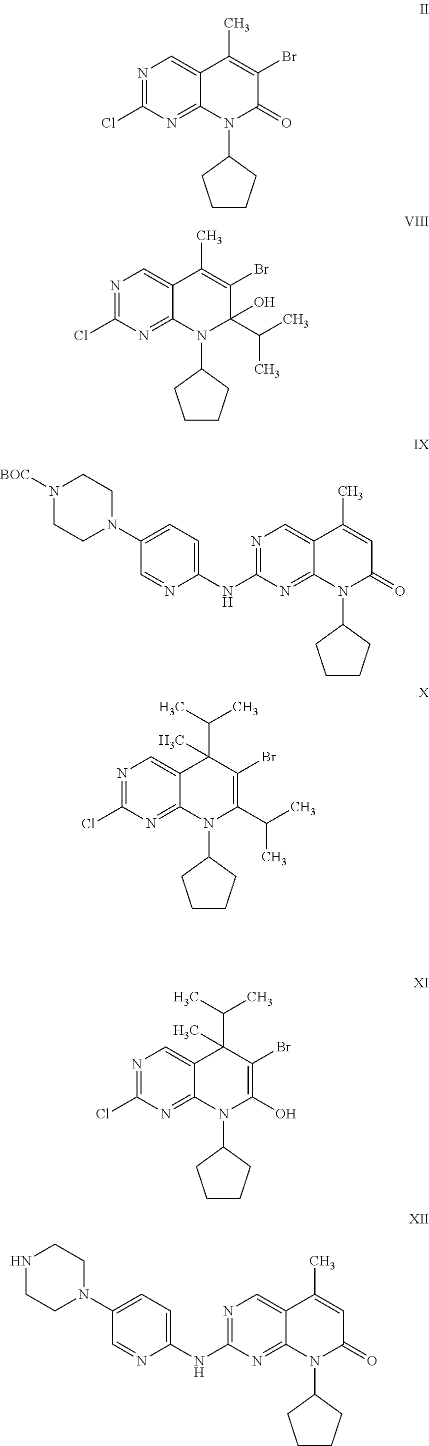

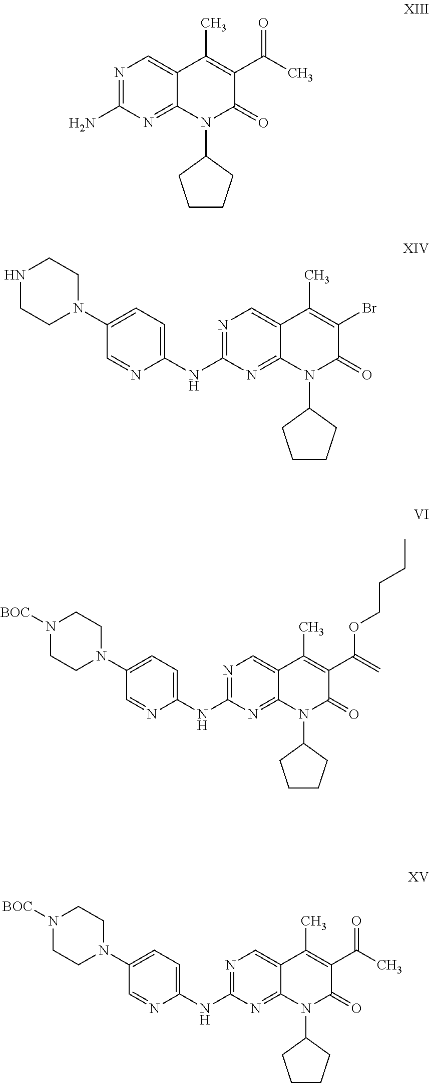 [SMILES: CC(=O)c1c(C)c2cnc(N)nc2n(C2CCCC2)c1=O ; Cc1c(Br)c(=O)n(C2CCCC2)c2nc(Nc3ccc(N4CCNCC4)cn3)ncc12 ; C=C(OCCCC)c1c(C)c2cnc(Nc3ccc(N4CCN(C(=O)OC(C)(C)C)CC4)cn3)nc2n(C2CCCC2)c1=O ; CC(=O)c1c(C)c2cnc(Nc3ccc(N4CCN(C(=O)OC(C)(C)C)CC4)cn3)nc2n(C2CCCC2)c1=O]

In one embodiment, the present invention provides a pharmaceutical composition comprising palbociclib (the “compound I”), wherein the compound I is prepared using crystalline Form G of N-BOC palbociclib, wherein the content of the compound VII, the compound VIII and the compound IX is less than 1% w/w as measured by HPLC.

In one embodiment, the present invention provides crystalline palbociclib characterised by X-ray powder diffraction pattern (XRPD) having peaks at about 5.0, 10.0, 10.2, 11.4, 17.0, 19.6 and 22.±0.2 degrees 2 theta.

In one embodiment, the isolated palbociclib has a purity ≥99% as measured by HPLC (high performance liquid chromatography).

In one embodiment, the isolated palbociclib has a purity ≥99.7% as measured by HPLC.

In an embodiment, the palbociclib contained in the pharmaceutical composition of the present invention is having D₉₀ particle size of less than about 30 microns, preferably less than about 25 microns; wherein the palbociclib is obtained by the process of the present invention.

In one embodiment, the present invention provides pharmaceutical compositions comprising crystalline palbociclib obtained by the processes herein described.

The examples that follow are provided to enable one skilled in the art to practice the invention and are merely illustrative of the invention. The examples should not be read as limiting the scope of the invention.

### EXAMPLES

### General Methods:

### 1. HPLC Method:

### For Related Substances:

High performance liquid chromatography (HPLC) was performed with the conditions described below for detecting chemical purity:

Column: Waters Symmetry Shield RP 18 5 μm 150×4.6; Column Temperature: 30° C., Mobile phase: Mobile Phase A: 0.01M Sodium Perchlorate in water adjusted pH=3.0 with Perchloric acid; Mobile Phase B: Acetonitrile; Sample Cooler temperature: 5° C.

| Time (min) | % Mobile Phase A | % Mobile Phase B |
|---|---|---|
| 0.01 | 85 | 15 |
| 05 | 75 | 25 |
| 20 | 45 | 55 |
| 25 | 25 | 75 |
| 34 | 25 | 75 |
| 35 | 85 | 15 |
| 39 | 85 | 15 |

Diluent: Buffer: Methanol (70:30, v/v); Buffer: 0.1% Glacial acetic acid in Water; Flow Rate: 1.0 mL/Minute, Detection wavelength: UV 260 nm; Injection Volume: 20 μL; Run time: 39 minutes.

### For Assay:

Mobile Phase: Buffer: Acetonitrile (75:25); Gradient Program: Isocratic, Diluent Solution A: 0.1% Orthophosporic Acid.

### 2. X-Ray Powder Diffraction (XRPD) (Instrumental Settings):

The measurements were performed on Philips X-Ray Diffractometer model XPERT-3 (PANalytical) Detector: X′celerator using Cu lamp with type and wavelength of the X-ray radiation: K-α₁ 1.54060[Å], K-α₂ 1.5444[Å] under the following conditions:

The measurements were carried out with a Pre FIX module programmable soller slit and anti-scatter Slit (Offset 0.00°); Generator settings: 40 mA/45 kV, tube current 40 mAmp Time per step: 50 s, Step size: 0.0167, Peak width 2.00 and start angle (°) 2.0 and End angle: 50.0; Scan type: continuous; measurement performed at 25° C. The XRPD instrument is calibrated using NIST SRM 640C silicon standard and NIST SRM 1976b Alumina.

Sample preparation: Take an adequate amount of the sample to fill the sample holder using back-loading technique. Then load the sample holder between the X-ray optics-path and scan using the above described parameters. Integrate the obtained powder X-ray diffraction profiles using HighScore Software.

### 3. Particle Size Distribution

The measurements were performed on Malvern 3000 Hydro MV with Laboratory Ultrasonicator (Output Voltage: 230V/50 Hz).

Instrumental Settings: Material R.I.: 1.480; Material absorption: 1.0; (Same parameters for blue light); Dispersant Name: 0.5% Span-85 in n-Hexane; Particle Absorbance Index: 0.00 Dispersant R.I.: 1.380; Model: General purpose; Scattering Model: Mie, Sensitivity: Normal; Particle shape: Non spherical (Irregular); Measurement Time: 10.0 secs; Background Time: 10.0 secs; Obscuration range: 10-20%; Stirrer speed: 2200 rpm; Measurement repeat: 3 times at zero second interval; report average (instrument averaged) results.

Sample preparation: Transfer about 50 mg of sample in glass beaker and add 25-30 mL of dispersant into it. Shake gently with glass rod and ensure complete dispersion of the sample into dispersant. Sonicate the suspension for 60 second with continuous stirring. After sonication again ensure dispersion of the sample into dispersant by gentle shaking. Add sample gently into the sampler accessory with the help of dropper.

### Example 1: 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester [compound IV]

To a suspension of tert-butyl 4-(6-amino-3-pyridyl)piperazine-1-carboxylate (24.32 gm, compound III) and toluene (125 mL) was added isopropyl magnesium chloride solution in tetrahydrofuran (THF) (80 mL) under nitrogen atmosphere at a temperature of 20° C. to 25° C. in 30 min. The resulting clear reaction mixture was stirred at a temperature of 20° C. to 25° C. for 30 min. 6-bromo-2-chloro-8-cyclopentyl-5-methylpyrido [2, 3-d]pyrimidin-7(8H)-one (25 gm, compound II) was added in four equal portions in the reaction mixture at a temperature of 20° C. to 30° C. over a period of 15 min. The reaction mixture was stirred for a period of 90 min at a temperature of 20° C. to 25° C. Methanol was added to the reaction mixture followed by addition of acetic acid at a temperature of 20° C. to 30° C. The reaction mixture was stirred for 2 hours at a temperature of 20° C. to 30° C. The product was filtered and dried in vacuum at a temperature of 55° C. to 65° C. to obtain 36.2 gm of compound IV.

Yield: 85.17%; Purity: 98.02% (as determined by HPLC).

Compound VIII: Not detected; des-bromo impurity IX: 0.40%; compound X and Compound XI: Not detected.

### Example 2: tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate[N-BOC palbociclib, compound VI]

In a round bottom flask, n-butanol (250 mL), compound IV (50 gm), n-butylvinylether (25.7 gm, compound V), diisopropylethylamine (55.28 gm), Bis[(2-diphenylphosphino)phenyl]ether (0.92 gm) and [1,1′-Bis(diphenylphosphino)ferrocene]palladium (II) dichloride (313 mg) were charged at a temperature of 20° C. to 30° C. to obtain a reaction mixture. The temperature of the reaction mixture is then raised to 105° C., and stirred for 16 hours. The reaction mixture was cooled to a temperature of 70° C., and n-butanol and water were added to it. Further, the reaction mixture was stirred for 20 min. The reaction mixture was filtered. Water was added, followed by addition of 1,2-diaminopropane (31.5 gm). The reaction mixture was stirred for 30 min at a temperature of 70° C. Aqueous layer was separated followed by distillation of n-butanol layer under vacuum at a temperature of 65° C. Ethyl acetate was added to the obtained concentrated mass, and then the reaction mixture was heated to a temperature of 70° C. The reaction mixture was filtered hot, and the filtrate was cooled to a temperature of 30° C., and further cooled to a temperature of 5° C. The resulting solid was stirred at a temperature of 5° C. for 3 hours and filtered. The wet cake was treated with L-cysteine in ethyl acetate and heated to obtain a clear solution. The clear solution was filtered and the filtrate was cooled. The solid obtained was dried in a vacuum tray drier (VTD) at a temperature of 65° C. to 70° C.

Yield: 41.2 gm; Purity: 99.27% (as determined by HPLC), des-bromo impurity IX: 0.05%.

### Example 3: Process for Palbociclib

In a round bottom flask N-BOC palbociclib (compound VI) (50 gm), water (500 mL) and acetone (250 mL) were charged, and heated to a temperature of 65° C. to 70° C. to obtain a reaction mixture. Gradually, conc. HCl (50.0 gm) was added to the reaction mixture over a period of not less than 4 hours at a temperature of 65° C.-70° C. The reaction was stirred for a period of 4 hours to 7 hours. On completion, the reaction mixture was cooled to a temperature of 25° C. to 30° C. and added 5% sodium hydroxide solution in portions to the reaction mixture to adjust the pH between 9 to 11. The reaction was stirred for a period of 1 hour to 2 hours at a temperature of 25° C. to 30° C. The precipitated product was filtered, and dried under vacuum to obtain 36.5 gm of palbociclib.

Yield: 90 to 95%; Purity: 99.90% (as determined by HPLC);

Compound VIII; Not detected; des-bromo impurity (IX): below 0.05%; compound (X) and compound XI: Not detected.

### Reference Example 1. 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester (compound IV) (WO 2014/128588A1, Example 3)

To a suspension of tert-butyl 4-(6-Amino-3-pyridyl) piperazine-1-carboxylate (52.83 g, compound III) and tetrahydrofuran (750 mL) was added isopropyl magnesium chloride solution 2M in THF (77 mL) under nitrogen atmosphere at a temperature of 20° C. in 30 min. The resulting clear reaction mixture was stirred at a temperature of 20° C. for 60 min. 6-bromo-2-chloro-8-cyclopentyl-5-methylpyrido [2,3-d]pyrimidin-7(8H)-one (50 g, compound II) added all at once in reaction mixture at a temperature of 20° C. and rinsed with THF. Again add isopropyl magnesium chloride solution 2M in THF (77 ml) was added under nitrogen atmosphere at a temperature of 20° C. in 30 min. Stirred the reaction mixture for 90 min followed by increase in the reaction temperature of 20° C.-60° C.

After completion, the reaction mixture was quenched with a mixture of THF and AcOH. The reaction mixture was filtered and washed with a mixture of acetone-water and again with acetone. The wet cake obtained was dried in VTD at 65° C.

Yield: 55 g (64.47%); Purity: 97.79% (as determined by HPLC)

Compound VIII: 0.08%; Des-bromo impurity (IX): 0.76%; compound X: 1.11%; compound XI: 0.20%.

### Comparative example 1: 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester (compound IV) (Org. Process Res. Dev. 2016, 20, 1191-1202 Example 4.1.2)

To a suspension of tert-butyl 4-(6-amino-3-pyridyl) piperazine-1-carboxylate (10.53 gm, compound III), and THF (50 mL) was added isopropyl magnesium chloride solution in THF (16 mL) under nitrogen atmosphere at a temperature of 10° C.-15° C. for 30 min to obtain a reaction mixture. The resulting clear reaction mixture was stirred to a temperature of 10° C. to 15° C. for 30 min. 6-bromo-2-chloro-8-cyclopentyl-5-methylpyrido [2, 3-d]pyrimidin-7(8H)-one (10 gm, compound II) was added in 2 lots in the reaction mixture at a temperature of 10° C. to 15° C. The reaction mixture was maintained for 10° C.-15° C. for 30 min. Again isopropyl magnesium chloride solution in THF (16 mL) was added under nitrogen atmosphere to the reaction mixture at a temperature of 10° C. to 15° C. in 30 min. The temperature of the reaction mass was raised to 20° C. The reaction mixture was stirred for 2 h at a temperature of 20° C. to −25° C. and then to a temperature of 55° C. for 1 hour. The reaction mixture was cooled to a temperature of 20° C.-25° C., and methanol was added to the reaction mixture followed by addition of acetic acid at a temperature of 20° C. to 30° C. The reaction mixture was stirred for 2 hours to 3 hours at a temperature of 20° C. to 30° C. The precipitated product was filtered. The solid obtained was dried in VTD at 55° C.-65° C.

Yield: 11.2 g (65.8%); Purity: 97.32% (as determined by HPLC),

compound VIII: 0.03%; des-bromo impurity IX: 0.35%; compound X: 1.29%; and Compound XI: 0.07%.

### Comparative Example 2: 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester (Compound IV)

To a suspension of tert-butyl 4-(6-Amino-3-pyridyl) piperazine-1-carboxylate (9.745 g, compound III), and toluene (50 mL) was added isopropyl magnesium chloride solution 2M in THF (32 mL) under nitrogen atmosphere at a temperature of 40° C.-45° C. in 30 min. The resulting clear reaction mixture was stirred at a temperature of 20° C.-25° C. for 30 min. 6-bromo-2-chloro-8-cyclopentyl-5-methylpyrido[2, 3-d]pyrimidin-7(8H)-one (10 g, compound II) was lot wise added in reaction mixture at a temperature of 20° C.-30° C. The reaction mixture was stirred for a period of 90 min at a temperature of 20° C.-25° C. Methanol was added to the reaction mixture followed by addition of acetic acid at a temperature of 20° C.-30° C. The reaction mixture was stirred at a temperature of 20° C.-30° C. for 3-4 hours, filtered and washed with methanol. The solid obtained was dried in VTD (Vacuum tray Dryer) at a temperature of 55-65° C.

Yield: 9.7 (56.89%); Purity: 90.94% (as determined by HPLC);

Compound VIII: Not detected; des-bromo impurity IX: 4.15%; compound X: Not detected; Compound XI: 0.06%.

### Comparative Example 3: 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester (Compound IV)

To a suspension of tert-butyl 4-(6-amino-3-pyridyl) piperazine-1-carboxylate (21.06 g, compound III), and toluene (100 mL) was added isopropyl magnesium chloride solution 2M in THF (32 mL) under nitrogen atmosphere at a temperature of 15° C.-25° C. in 30 min. The resulting clear reaction mixture was stirred at a temperature of 15° C.-25° C. for 30 min. 6-bromo-2-chloro-8-cyclopentyl-5-methylpyrido [2, 3-d]pyrimidin-7(8H)-one (20 g, compound II) was lot wise added to the reaction mixture at a temperature of 15° C.-25° C. The reaction mixture was maintained at a temperature of 20° C.-30° C. for 60 min. Isopropyl magnesium chloride solution 2M in THF (32 mL) was added under nitrogen atmosphere in reaction mixture at a temperature of 15° C.-25° C. in 30 min. The temperature of the reaction mixture was raised to a temperature of 20° C. The reaction mixture was stirred for 90 min at a temperature of 20° C.-25° C. Methanol was added to the reaction mixture followed by addition of acetic acid at a temperature of 20° C.-30° C. Stirred the reaction mixture for 2-3 hour at a temperature of 20° C.-30° C., filtered and washed with methanol. The solid obtained was dried in VTD at a temperature of 55° C.-65° C.

Yield: 27.29 (80%); Purity: 96.82% (as determined by HPLC);

Compound VIII: Not detected; des-bromo impurity IX: 2.13%; Compound X: Not detected; Compound XI 0.01%.

1. A process for the preparation of palbociclib, a compound of formula I, (the “compound I”), 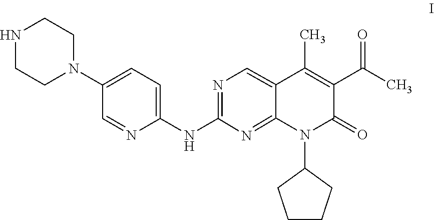 [SMILES: CC(=O)c1c(C)c2cnc(Nc3ccc(N4CCNCC4)cn3)nc2n(C2CCCC2)c1=O]
comprising the steps of:
a) reacting 6-bromo-2-chloro-8-cyclopentyl-5-methylpyrido[2,3-d]pyrimidin-7(8H)-one, a compound of formula II, (the “compound II”) with tert-butyl 4-(6-amino-3-pyridyl) piperazine-1-carboxylate, a compound of formula III (the “compound III”) in the presence of a Grignard reagent, 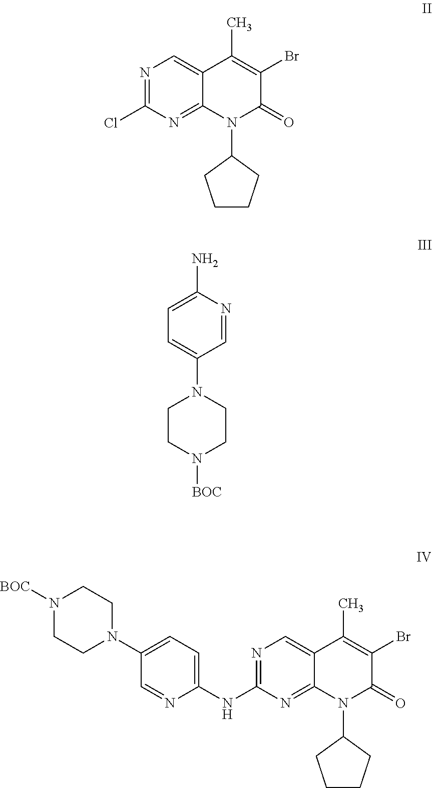 [SMILES: Cc1c(Br)c(=O)n(C2CCCC2)c2nc(Cl)ncc12 ; CC(C)(C)OC(=O)N1CCN(c2ccc(N)nc2)CC1 ; Cc1c(Br)c(=O)n(C2CCCC2)c2nc(Nc3ccc(N4CCN(C(=O)OC(C)(C)C)CC4)cn3)ncc12]
to obtain 4-{6-[6-bromo-8-cyclopentyl-5-methyl-7-oxo-7,8-dihydro-pyrido[2,3-d]pyrimidin-2-ylamino]-pyridin-3-yl}-piperazine-1-carboxylic acid-butyl ester, a compound of formula IV, (the “compound IV”), wherein the Grignard reagent is added in one lot, and the compound II is added in at least two portions;
b) reacting the compound IV obtained in the step a) with n-butyl vinyl ether, a compound of formula V, (the “compound V”) 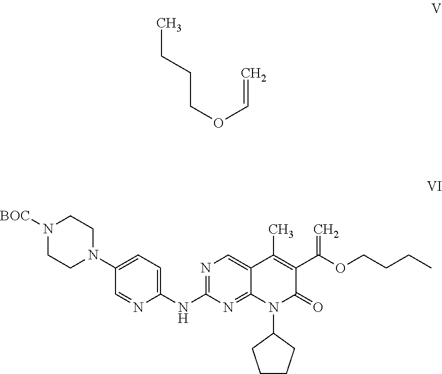
to obtain tert-butyl 4-(6-{[6-(1-butoxyethenyl)-8-cyclopentyl-5-methyl-7-oxo-7,8-dihydropyrido[2,3-d]pyrimidin-2-yl]amino}pyridin-3-yl)piperazine-1-carboxylate, a compound of formula VI (the “compound VI”); and
c) reacting the compound VI obtained in the step b) with an acid followed by treating with a base to obtain palbociclib, the compound I.

2. The process according to claim 1, wherein the step a) comprises the steps of:
(a-i) reacting the compound II with the compound III in a mixture comprising a solvent and a Grignard reagent, wherein less than or equal to one molar equivalent of the compound II is used with respect to compound III, and wherein the Grignard reagent is added in one lot;
(a-ii) stirring the reaction mixture of the step (a-i) for a period of about 15 minutes to about 1 hour;
(a-iii) adding second portion of the compound II to the mixture of step (a-ii) to obtain the compound IV;
(a-iv) optionally, repeating the step (a-ii) and the step (a-iii) to have total molar equivalent of compound II with respect to compound III stoichiometrically between 0.8 to 1.0 molar equivalents; and
(a-v) isolating the compound IV obtained in the step (a-iii) or the step (a-iv).

3. The process according to claim 2, wherein the step (a-i) comprises addition of the Grignard reagent at a temperature below 40° C.

4. A The process according to claim 1, wherein the compound VI is crystalline Form G, which is characterised by an X-ray powder diffraction (XRPD) pattern having peaks at about 5.87, 9.29, 10.52, 11.77, 12.97, 13.72, 16.31, and 22.44±0.2 degrees 2 theta.

5. A The process according to claim 1, wherein the compound VI is crystalline Form G, which is as substantially illustrated in FIG. 1.

6. A process for the preparation of crystalline Form G of the compound VI, wherein the process comprises the steps of:
(i) providing a solution of, the compound VI in an ester solvent;
(ii) heating the solution of the step (i);
(iii) optionally, filtering the hot solution of the step (ii);
(iv) cooling the solution of the step (ii) or the step (iii); and
(v) isolating the crystalline Form G of the compound VI, characterised by an XRPD pattern having peaks at about 5.87, 9.29, 10.52, 11.77, 12.97, 13.72, 16.31, and 22.44±0.2 degrees 2 theta.

7. A compound selected from:
a) a compound of formula VII (the “compound VII”), 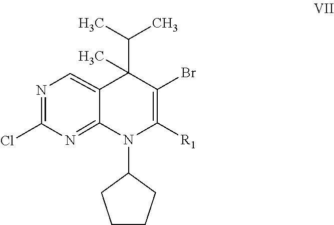
wherein R¹ is hydroxy or isopropyl; or
b) a compound of formula VIII (the “compound VIII”),

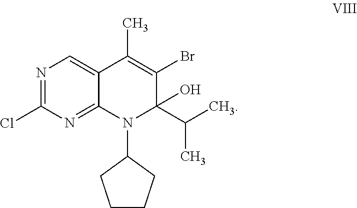 [SMILES: CC1=C(Br)C(O)(C(C)C)N(C2CCCC2)c2nc(Cl)ncc21]

8. (canceled)

9. The process according to claim 1, wherein the palbociclib represented by the compound of formula I, 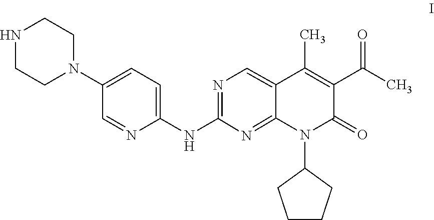 [SMILES: CC(=O)c1c(C)c2cnc(Nc3ccc(N4CCNCC4)cn3)nc2n(C2CCCC2)c1=O]
contains a compound VII, a compound VIII and a compound IX represented by the following structures: 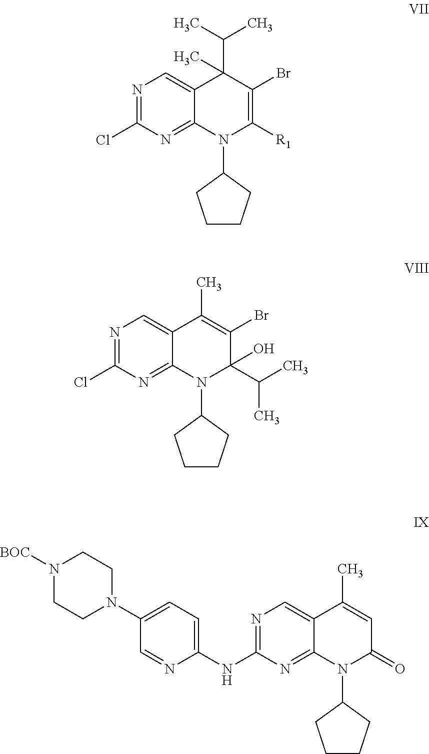
wherein R is hydroxy or isopropyl,
in an amount ranging from about 0.15% w/w to about 0.03% w/w as determined by High-performance liquid chromatography (HPLC).

10. (canceled)
